(19) United States
(12) Patent Application Publication
(10) Pub. No.: US 2005/0096516 A1
(43) Pub. Date: May 5, 2005

(54) Optical detector of organic analyte

(76) Inventors: Orhan Soykan, Shoreview, MN (US); Sheila A. Grant, Columbia, MO (US); Darcy J. Lichlyter, Columbia, MO (US)

Correspondence Address: SHUMAKER & SIEFFERT, P. A., 8425 SEASONS PARKWAY, SUITE 105, ST. PAUL, MN, 55125, US

(21) Appl. No.: 10/698,050

(22) Filed: Oct. 30, 2003

(51) Int. Cl. A61B 5/00

(52) U.S. Cl. 600/317

(57) ABSTRACT

The invention is directed to techniques for optically detecting changes in concentration of analytes in the body of a patient via fluorescent resonant energy transfer (FRET). An analyte detector implantable in the body of the patient includes a plurality of fluorophore-tagged sensing elements that bind to a specific analyte. A light emitter emits energy at a wavelength that is within the absorption spectrum of a donor fluorescent dye, and a light detector detects the energy fluoresced by donor and acceptor fluorescent dyes in the analyte detector. The relative intensity of energy fluoresced by the dyes is a function of the concentration of the analyte. A processor monitors the change in concentration of the analyte over time and may take action, such as directing the therapy device to administer therapy, when the change in concentration surpasses a predetermined threshold.

### TECHNICAL FIELD

The invention relates to medical devices, and more particularly, to medical devices that detect organic substances such as proteins within bodily fluid.

### BACKGROUND

The presence or concentration of a particular analyte in the body of a patient may have a clinical significance. An “analyte” is any substance of interest. Typical analytes include molecular or microscopic substances such as proteins or medications or viruses.

A patient suffering from acute myocardial infarction, for example, experiences a sudden increase in cardiac troponin-T antigen in his bloodstream as the heart cells undergoing necrosis release cardiac troponin-T. This relatively sudden increase in the concentration of cardiac troponin-T is a specific, reliable indicator of the acute myocardial infarction. Detection of the change of concentration of this analyte, therefore, may be of clinical significance to the patient. Detection of the change of concentration of this analyte may be pertinent to detection of a serious medical condition, and may also be pertinent to diagnosis and prompt treatment.

There are many other analytes that may be markers for other specific medical conditions. In many cases, the presence or absence of an analyte, or a sudden change in concentration of the analyte, accompany a significant health-related concern.

### SUMMARY

In general, the invention is directed to techniques for optically detecting changes in concentration of analytes in the body of a patient. In particular, the invention responds to analyte concentration by fluorescent resonant energy transfer (FRET).

An analyte detector implantable in the body of the patient includes a plurality of sensing elements that bind to the specific analyte in question. When the analyte is cardiac troponin-T antigen, for example, the sensing elements may be cardiac troponin-T antibodies, which bind to cardiac troponin-T antigen with high specificity. The sensing elements may be anchored to a substrate.

The sensing elements may be tagged with two fluorescent dyes, a donor and an acceptor. In a typical analyte detector, one dye may be coupled to a sensing element and the other may be coupled to the substrate or to an anchoring agent that anchors the sensing element to the substrate. The dyes fluoresce in a narrow range of wavelengths (their emission spectra) when they receive energy in another range of wavelengths (their absorption spectra). The absorption spectrum of the acceptor fluorescent dye overlaps the emission spectrum of the donor fluorescent dye.

The invention includes emitting energy toward the analyte detector at a wavelength that is within the absorption spectrum of the donor fluorescent dye. In response, the donor fluorescent dye fluoresces energy at a wavelength within its emission spectrum. When a sensing element is not bound to an analyte, the donor and acceptor fluorescent dyes are not in sufficient proximity such that emission of energy from the donor fluorescent dye will cause the acceptor fluorescent dye to fluoresce.

When a sensing element binds to an analyte, however, the sensing element undergoes a conformational change that brings the dyes into sufficient proximity to allow FRET to occur. When the dyes are sufficiently proximate and properly oriented, the emission of energy by the donor fluorescent dye causes the acceptor fluorescent dye to fluoresce. As a result, the energy emitted by a sensing element depends upon whether an analyte is bound to the sensing element or not.

A light detector receives the light fluoresced by the analyte detector, and a processor monitors the received light. In particular, the processor monitors the intensity of energy emitted in the emission spectrum of the donor fluorescent dye, in relation to the intensity of energy emitted in the emission spectrum of the acceptor fluorescent dye. The relative intensity of energy at these two wavelengths is a function of the number of sensing elements having analyte bound to them, which in turn is a function of the concentration of the analyte in the body of the patient.

In many cases, the change in concentration of the analyte over time is of clinical significance. When a patient suffers from acute myocardial infarction, for example, the patient experiences a sudden increase in concentration of cardiac troponins, such as cardiac troponin-T and cardiac troponin-I, in his body. It is the abrupt increase in concentration of cardiac troponin-T that is indicative of an onset of myocardial infarction. The processor may therefore compare changes in relative concentration to a predetermined threshold, and may take action when the threshold is surpassed. Taking action may include notifying a therapy device or directing the therapy device to administer therapy. Taking action may also involve generating an alert to notify the patient or another person about a potentially serious health condition.

In one embodiment, the invention is directed to a method. The method includes emitting energy at a first wavelength (which will be referred to below as λ₀) toward a detector implanted in a body of a patient. The detector includes a binding site for an analyte, and also includes at least two fluorescent dyes. The method further includes detecting energy emitted by a first one of the dyes (an acceptor fluorescent dye) at a second wavelength (which will be referred to below as λ₂), based on fluorescent resonant energy transfer from a second one of the dyes (a donor fluorescent dye) in response to the emitted energy at the first wavelength. The method further includes detecting a change in concentration of the analyte as a function of the detected energy.

The method may further include comparing the detected change to a threshold, and taking action when the detected change surpasses the threshold. The method may further include detecting energy at a third wavelength (which will be referred to below as λ₁) emitted by the second one of the dyes (a donor fluorescent dye) in response to the emitted energy at the first wavelength.

In another embodiment, the invention is directed to a computer-readable medium containing instructions that cause a programmable processor to carry out the methods of the invention.

In a further embodiment, the invention is directed to a system comprising a light emitter to emit energy at a first wavelength. The system also comprises an analyte detector implanted in a body of a patient. The analyte detector includes at least one binding site for an analyte and at least two fluorescent dyes. The system further comprises a light detector to detect energy at a second wavelength emitted by a first one of the dyes based on fluorescent resonant energy transfer from a second one of the dyes in response to the emitted energy at the first wavelength. The system also comprises a processor to detect a change in a concentration of the analyte as a function of the energy detected by the light detector. The system may further include a therapy device to deliver therapy to the patient based on the detection of the change in a concentration and an alert module to notify the patient based on the detection of the change in a concentration.

In an additional embodiment, the invention is directed to a device comprising a substrate, at least two fluorescent dyes and a plurality of sensing elements anchored to the substrate. Each sensing element includes a binding site for an analyte, and each sensing element is configured to bring the dyes into proximity to allow fluorescent resonant energy transfer when the analyte binds to the binding site.

The various embodiments of the invention may provide one or more advantages. The sensing elements can be highly specific to a particular analyte, and can be very sensitive to changes in concentration of the analyte. In addition, a fluorescence-based analyte detector may undergo some encapsulation by the body following implantation, but fluorescence-based detection is less sensitive to problems associated with encapsulation than other detection techniques.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic diagram illustrating a device for optically detecting the concentration of an analyte in a body of a patient.

FIG. 2 is a conceptual diagram an implantable analyte sensor.

FIG. 3 is a conceptual diagram an implantable analyte sensor, comprising an implantable portion and an external portion.

FIGS. 4A and 4B are conceptual diagrams of an exemplary sensing element, illustrating detection of an analyte with FRET.

FIG. 5 is a conceptual perspective diagram of an analyte detector.

FIG. 6 is a flow diagram illustrating techniques for monitoring changes in analyte concentration.

FIG. 7 is a timing diagram illustrating a curve of relative intensity of energy from an analyte detector over time.

FIG. 8 is a bar chart illustrating specificity of analyte detection with FRET.

### DETAILED DESCRIPTION

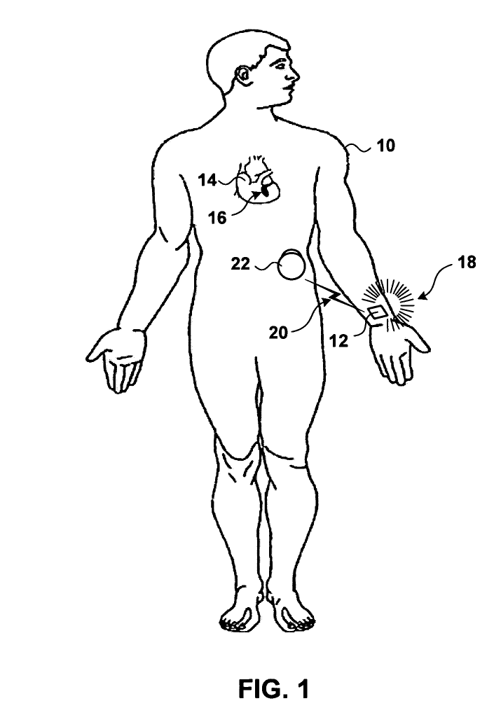

FIG. 1 is a diagram illustrating a patient 10 with an analyte sensor 12. Sensor 12 may be implanted in the body of patient 10, or may include a portion external to the body of patient 10. For purposes of illustration of the invention, sensor 12 will be described as responsive to changes in concentration of a specific protein, cardiac troponin-T antigen. The invention is not limited to detection of cardiac troponin-T, however, but may be adapted to detect other organic analytes as well.

The heart 14 of patient 10 is susceptible to coronary artery disease, in which formation of plaques narrows the lumen of one or more vessels supplying 02 to cardiac tissue. If the vessels become occluded, the cardiac tissue 16 served by the vessel soon dies from O₂ deprivation. Actual necrosis of heart tissue is called acute myocardial infarction, or heart attack. The necrosis of heart tissue is accompanied by the release of cardiac troponin-T into the bloodstream of patient 10. Sensor 12 detects the sudden increase in the concentration of cardiac troponin-T antigen, which is a reliable indicator of myocardial infarction.

Patient 10 may have sensor 12 when the physician for patient 10 deems that patient 10 is at risk of myocardial infarction. Patient 10 may be deemed at risk of myocardial infarction when, for example, patient 10 has had a previous episode of myocardial infarction, or when patient 10 has a history of coronary artery disease, or when patient 10 suffers from diabetes.

In response to the detection of a sudden increase in the concentration of cardiac troponin-T, sensor 12 can generate an alert 18 that notifies patient 10 of the onset of infarction. In the embodiment of the invention depicted in FIG. 1, sensor 12 can further generate a wireless communication 20 to a therapy device 22. Therapy device 22 may be implanted in the body of patient 10 or may be external. In another embodiment of the invention, sensor 12 is combined with or physically coupled to therapy device 22, and both sensor 12 and therapy device 22 are implanted in the body of patient 10. In this embodiment, sensor 12 may communicate with therapy device 22 via a communication technique other than wireless communication.

Therapy device 22 delivers therapy to address the condition of concern. Therapy device 22 may be any device that delivers any therapy. In the case of myocardial infarction, therapy device 22 may comprise an implanted drug delivery device that injects a thrombolytic agent into the bloodstream of patient 10. Therapy device 22 may also comprise an electrical stimulator that stimulates an organ, muscle or nerve to cause the release of a natural thrombolytic agent, such as tissue plasminogen activator (tPA). Therapy device 22 may also comprise a pacemaker that adjusts pacing therapy by, for example, slowing the pacing rate to reduce cardiac demand or by changing pacing mode.

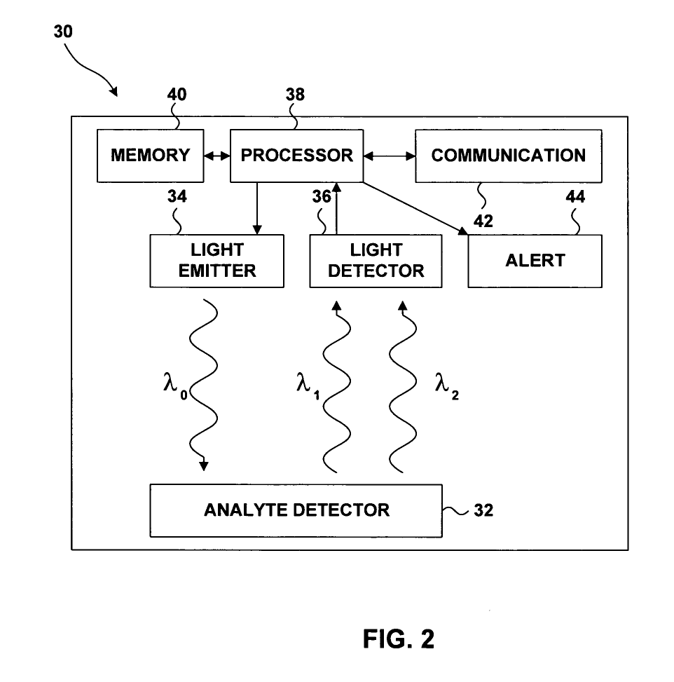

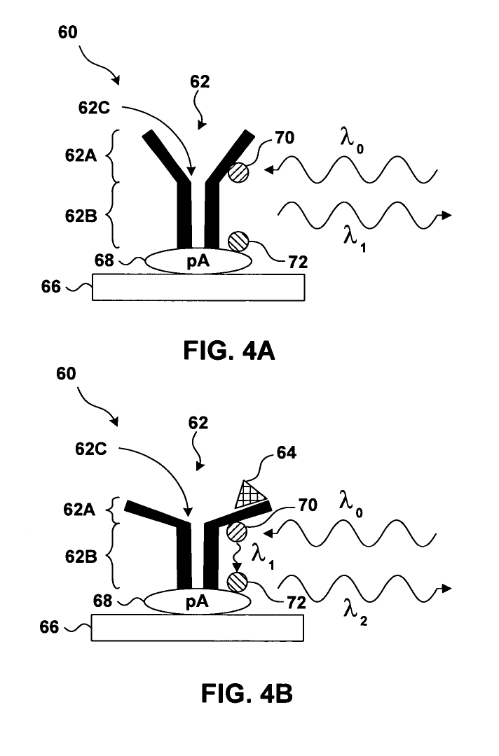

FIG. 2 is a block diagram showing an embodiment of an analyte sensor 30. The embodiment of a sensor 30 shown in FIG. 2 may be implantable in the human body. Sensor 30 includes an analyte detector 32, which is typically implanted in the body and which responds to the presence of the analyte of interest. As will be illustrated in FIGS. 4A and 4B, an analyte detector includes one or more sensing elements, such as antibodies, anchored to a substrate. Each sensing element includes a binding site that binds to a specific analyte of interest. In an illustrative embodiment, the sensing elements comprise troponin-T antibodies that bind selectively to cardiac troponin-T. When a sensing element binds to an analyte, the sensing element undergoes a conformational change, which may be detected optically by fluorescent resonant energy transfer (FRET), as described below.

Sensor 30 includes a light emitter 34 that emits energy at a wavelength λ₀ toward analyte detector 32. Light emitter 34 may comprise, for example, a laser diode that emits visible light in a narrow range of wavelengths around wavelength λ₀. In response, analyte detector 32 fluoresces energy at wavelengths λ₁ and λ₂.

The fluoresced energy strikes light detector 36. Light detector 36 can include one or more electro-optical transducers. In some embodiments, a single transducer detects energy at wavelengths λ₁ and λ₂. In such embodiments, light detector 36 can include analog or digital signal processing components in addition to an electro-optical transducer to determine the intensities at wavelengths λ₁ and λ₂. Light detector 36 may also include one or more amplifiers to amplify the received energy, and one or more filters, such as dichroic band-pass filters, to improve detection of energy at particular wavelengths.

In addition to one or more amplifiers or filters, light detector 36 typically includes circuitry to enhance detection of energy at wavelengths λ₁ and λ₂. In one embodiment of the invention, the energy emitted by light emitter 34 may be modulated by one or more chopper-stabilized amplifiers. Light detector 36 may likewise be coupled to the same timer circuit that regulates light emitter 34, thereby enhancing time coordination of emission and detection. By use of one or more chopper stabilized amplifiers, the energy emitted by light emitter 34 at wavelength λ₀ turns on and off during emission, becoming time varying at a known rate. Light detector 36 detects energy at wavelengths λ₁ and λ₂ that turn on and off at the same rate. In this way, light detector 36 is better able to distinguish meaningful emissions at wavelengths λ₁ and λ₂ from background noise, thereby improving the signal-to-noise ratio. In another embodiment of the invention, lock-in amplifiers may be used in place of chopper-stabilized amplifiers to improve the signal-to-noise ratio. A processor 38 may control light emitter 34 and light detector 36 to improve noise rejection and to conserve power.

The intensities of the energy at wavelengths λ₁ and λ₂, as detected by light detector 36, are supplied to processor 38. In some embodiments of the invention, processor 38 employs digital filtering or other noise rejection techniques to improve the signal-to-noise ratio. Processor 38 determines the relative intensity of the energy at wavelengths λ₁ and λ₂ with respect to one another by determining a ratio between the detected intensity of energy at wavelengths λ₁ and λ₂. Processor 38 can store the determined ratio in a memory 40.

Processor 38 may include one or more microprocessors, digital signal processors (DSPs), application specific integrated circuits (ASICs), field-programmable gate arrays (FPGAs), or the like. Memory 40 may include a variety of magnetic, optical, or electronic media, such as random access memory (RAM), read-only memory (ROM), electronic erasable programmable read-only memory (EEPROM), flash memory, or the like.

In addition to determining the relative intensity at a particular point in time, processor 38 analyzes the change in relative intensity over time. Processor 38 may, for example, take the derivative of the ratio with respect to time, or may otherwise monitor the change in the ratio. In many cases, the change in relative intensity with respect to time is a quantity of greater clinical significance than the relative intensity itself.

For example, a patient suffering an acute myocardial infarction experiences a relatively abrupt release of cardiac troponin-T into the bloodstream. This relatively sudden increase in the concentration of cardiac troponin-T is an indicator of the acute myocardial infarction. As the higher concentration of cardiac troponin-T encounters analyte detector 32, the relative intensities of the energy fluoresced by analyte detector 32 at wavelengths λ₁ and λ₂ changes rapidly. Processor 38 detects the rapid change in relative intensity, and in this way, detects the myocardial infarction.

Upon detection of a condition of interest, processor 38 may take action. In response to detection of a myocardial infarction, for example, processor 38 may notify therapy device 22, or may direct therapy device 22 to administer therapy, via a communication module 42. As noted above, the generated communication may be wireless, but the invention encompasses communication by other media as well.

Processor 38 may further take action by notifying patient 10. In some cases, including some cases of myocardial infarction, patient 10 may be unaware of his condition. Accordingly, sensor 30 may include an alert module 44 that notifies patient 10 of his condition or otherwise directs patient 10 to seek medical help. Alert module 44 may use any visual, tactile, auditory or other technique to notify patient 10.

Sensor 30 may be implanted by administering general or local anesthesia to patient 10, incising the skin, and creating a pocket for sensor 30.

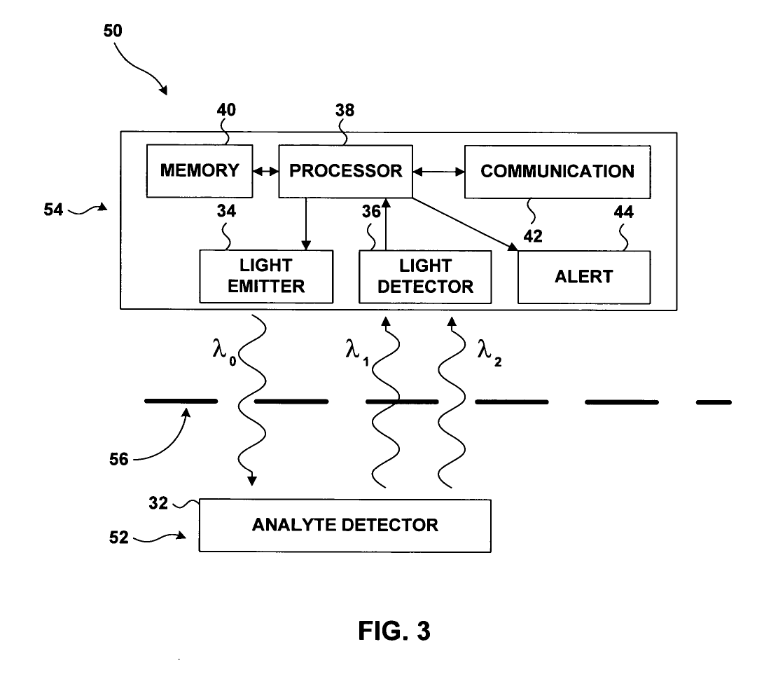

FIG. 3 is a block diagram showing an alternate embodiment of an analyte sensor 50. In the embodiment shown in FIG. 3, sensor comprises an implantable portion 52, which is implantable in the human body, and an external portion 54, which is not implantable. In the embodiment shown in FIG. 3, implantable portion 52 includes the analyte detector 32. External portion 54 includes light emitter 34, light detector 36, processor 38, communication module 42 and alert module 44.

Implantable portion 52 is deployed close to the surface of the skin 56 of patient 10, and is deployed at a site where skin 56 is translucent. A physician may choose any of several techniques for implanting implantable portion 52. When implantable portion 52 comprises one or more matchstick-size rods, for example, the rods may be inserted under the skin in a matter of minutes. Patient 10 typically receives a local anesthetic, and the physician injects or inserts the rods under skin 56 through one or more incisions. External portion 54 is deployed proximate to implantable portion 52, and may be held in place by any fixation mechanism, such as a fastener, belt or adhesive.

Light emitter 34 emits energy at a wavelength λ₀, which transits skin 56 and strikes analyte detector 32. In response, analyte detector 32 fluoresces energy at wavelengths λ₁ and λ₂. The fluoresced energy transits skin 56 and strikes light detector 36. The intensities of the energy at wavelengths λ₁ and λ₂ are supplied to a processor 38, which determines the relative intensity of the energy at wavelengths λ₁ and λ₂ with respect to one another by determining a ratio between the detected intensity of energy at wavelengths λ₁ and λ₂. Processor 38 also analyzes the change in relative intensity over time, as described above. Processor 38 further may take action in response to detection of a condition of interest. Such as communicating with therapy device 22 via communication module 42 or notifying patient 10 via alert module 44.

Embodiments of sensors 30 and 50 in FIGS. 2 and 3 may each have respective advantages. A fully implantable sensor such as sensor 30 in FIG. 2 is less likely to be misplaced, forgotten or damaged. Also, with a fully implantable sensor, less energy is lost as the emitted and detected energy transits tissue. An advantage of the partially implantable sensor, such as sensor 50, is that the surgical implantation procedure may be more convenient and less painful to patient 10. Removal or replacement of a set of analyte detectors may be easier than removal or replacement of a complete sensor.

FIGS. 4A and 4B illustrate one embodiment of a single exemplary sensing element 60. An analyte sensor may include a plurality of such sensing elements. Sensing element 60 includes a component that changes conformation in the presence of an analyte of interest. In exemplary sensing element 60 shown in FIGS. 4A and 4B, an antibody molecule 62 changes conformation in the presence of an analyte of interest 64. Furthermore, antibody molecule 62 is specific, changing conformation in the presence of analyte 64 but not changing conformation in the presence of other substances.

An exemplary antibody molecule 62 may be troponin-T antibody and an exemplary analyte 64 may be the cardiac troponin-T antigen. Troponin-T antibody binds selectively with cardiac troponin-T antigen, and changes conformation upon such binding.

Antibody molecule 62 is anchored to a substrate 66 by an anchoring agent. In the embodiment shown in FIGS. 4A and 4B, the anchoring agent is Protein A (pA) 68. Protein A 68 is a stable surface receptor produced by Staphylococcus Aureus (Staph A), and it binds with affinity to the nonreactive portions of certain immunoglobulins. In some embodiments of the invention, antibody molecule 62 may be anchored to substrate 66 by an anchoring agent other than pA 68, such as a bi-functional cross linker. A bi-functional cross linker includes at least two coupled groups, one of which binds to substrate 66 and the other of which binds to antibody molecule 62. In further embodiments, antibody molecule 62 may be anchored to substrate 66 without an anchoring agent.

Substrate 66 may be any biocompatible material, such as silicone or glass. Substrate 66 may be formed in any shape, such as a planar slide or a cylindrical fiber.

Antibody molecule 62 is a Y-shaped molecule that can assume at least two distinct conformations. In particular, antibody 62 includes an epitope or antigen binding site region 62A and a crystalline or stem region 62B. A hinge region 62C couples epitope region 62A to crystalline region 62B. As shown in FIG. 4A, antibody molecule 62 assumes a first conformation when analyte 64 is not present, and as shown in FIG. 4B, antibody molecule 62 assumes a second conformation when analyte 64 is present. In particular, the presence of analyte 64 causes epitope region 62A to move into a different orientation with respect to crystalline region 62B.

Sensing element 60 also includes a donor fluorescent dye 70 coupled to epitope region 62A of antibody molecule 62, and an acceptor fluorescent dye 72 coupled to Protein A 68. A sensing element tagged with donor and acceptor fluorescent dyes may be called “fluorophore-tagged.”

In FIG. 4A, energy at a wavelength λ₀, emitted by a light emitter 34, strikes donor fluorescent dye 70. In response, donor fluorescent dye 70 fluoresces energy at wavelength λ₁. Because of the conformation of antibody molecule 62, donor fluorescent dye 70 is not sufficiently proximate to acceptor fluorescent dye 72 to allow FRET to take place. Accordingly, when analyte 64 is not present, sensing element 60 receives energy at wavelength λ₀ and fluoresces energy at wavelength λ₁.

In FIG. 4B, energy at a wavelength λ₀, emitted by a light emitter 34, strikes donor fluorescent dye 70. In response, donor fluorescent dye 70 fluoresces energy at wavelength λ₁. Because of the presence of analyte 64, antibody molecule 62 in FIG. 4B has undergone a conformational change, bringing donor fluorescent dye 70 sufficiently proximate to acceptor fluorescent dye 72 to allow FRET to take place. In particular, when donor fluorescent dye 70 and acceptor fluorescent dye 72 are between approximately 10 and 100 Angstroms, and their dipole orientations are approximately parallel, acceptor fluorescent dye 72 will resonantly receive energy at wavelength λ₁ from donor fluorescent dye 70 via FRET.

In response to receiving energy at wavelength λ₁ from donor fluorescent dye 70, acceptor fluorescent dye 72 emits energy at wavelength λ₂. As shown in FIG. 4B, donor fluorescent dye 70 and acceptor fluorescent dye 72 are sufficiently proximate and properly oriented, such that acceptor fluorescent dye 72 emits energy at wavelength λ₂. Accordingly, when analyte 64 is present, sensing element 60 receives energy at wavelength λ₀ and fluoresces energy at wavelength λ₂. In addition, sensing element 60 fluoresces less energy at wavelength λ₁ when analyte 64 is present.

In one embodiment of the invention, donor fluorescent dye 70 comprises fluorescein 5-isothiocyanate (FITC). Receiving energy at a wavelength λ₀=494 nm, FITC fluoresces energy at wavelength λ₁=516-525 nm (or about 520 nm). In this embodiment, acceptor fluorescent dye 72 comprises tetramethylrhodamine 5 (and 6)-isothiocyanate (TRITC). Receiving energy at a wavelength λ₁=516-525 nm, TRITC fluoresces energy at wavelength λ₂=570-580 nm (or about 574 nm). TRITC does not substantially fluoresce in response to energy received at wavelength λ₀=494 nm, because this wavelength is outside the excitation spectrum of TRITC.

In this embodiment, analyte detector 32 (shown in FIGS. 2 and 3) comprises millions of fluorophore-tagged sensing elements similar to sensing element 60. Emitter 34 emits energy at a wavelength λ₀=about 494 nm toward analyte detector 32. In response, analyte detector 32 fluoresces energy at wavelengths λ₁=about 520 nm and λ₂=about 574 nm. Detector 36 receives the fluoresced energy and responds to the fluoresced energy as a function of the intensity of the energy at wavelengths λ₁ and λ₂.

The relative intensity of the energy at wavelengths λ₁ and λ₂ with respect to one another is a function of the concentration of analyte 64 in contact with analyte detector 32. Sensing elements that bind to analyte 64 fluoresce at wavelength 2, and sensing elements that do not bind to analyte 64 fluoresce at wavelength λ₁. A higher concentration of analyte 64 causes more energy to be transferred between dyes 70 and 72 by FRET, resulting in a higher intensity of energy at wavelength 2 relative to energy at wavelength λ₁.

Processor 38 receives the detected intensities and determines the relative intensity of the energy at wavelengths λ₁ and λ₂ with respect to one another by determining a ratio between the detected intensity of energy at wavelengths λ₁ and λ₂. This ratio is a function of the concentration of analyte 64 in contact with analyte detector 32. Processor 38 also analyzes the change in relative intensity over time, which is a function of the change in concentration of analyte 64 in contact with analyte detector 32.

The illustrated embodiment employs FITC as donor fluorescent dye 70 and TRITC as acceptor fluorescent dye 72, but the invention is not limited to these dye pairs. Suitable dye pairs include any dye pair that has an acceptor with absorption spectrum that overlaps the emission spectrum of the donor. For example, another possible dye pair may include aminomethylcoumarin acetate (AMCA) as the donor fluorescent dye and FITC as the acceptor fluorescent dye. The average wavelength of energy emitted by emitter 34 will ordinarily depend upon the dye pair that is employed. Suitability of a particular dye pair may also depend on its ability to bond to a particular substrate or a particular sensing element.

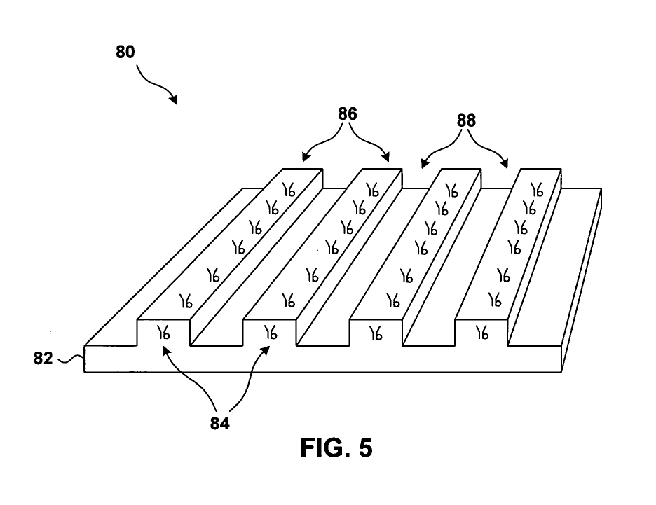

FIG. 5 is a view of an alternate embodiment of analyte detector 32. In the embodiment depicted in FIG. 5, analyte detector 80 comprises a substrate 82 and a plurality of fluorophore-tagged sensing elements 84. Fluorophore-tagged sensing elements 84, which are represented as antibody molecules, are anchored directly to substrate 82. In other words, fluorophore-tagged sensing elements 84 are anchored to substrate 82 without an anchoring agent such as Protein A. Rather, fluorophore-tagged sensing elements 84 are anchored to substrate 82 by being enclosed in substrate 82. Donor or acceptor dyes may be anchored to substrate 82 rather than sensing elements 84.

Although substrate 82 anchors fluorophore-tagged sensing elements 84, substrate 82 is permeable to the analyte of interest. Accordingly, the analyte can permeate substrate 82 and bind to a fluorophore-tagged sensing element 84, thereby changing the conformation of fluorophore-tagged sensing element 84 and enhancing energy transfer by FRET. Various hydrogels or matrices of polytetrafluoroethylene (PTFE) may be used to construct substrate 82.

In the embodiment depicted in FIG. 5, substrate 82 includes ridges 86 and grooves 88. Ridges 86 and grooves 88 enhance vascularization that may take place upon implantation of analyte detector 80. Vascularization may impede the passage of light but enhance the detection of the analyte, thus vascularization may improve the operation of analyte detector 80. The width of a typical ridge 86 or groove 88 may about one to three micrometers, and a groove 88 may have a depth of about one-half to one micrometer. In one embodiment, the overall dimensions of analyte detector 80 can be one centimeter by one centimeter.

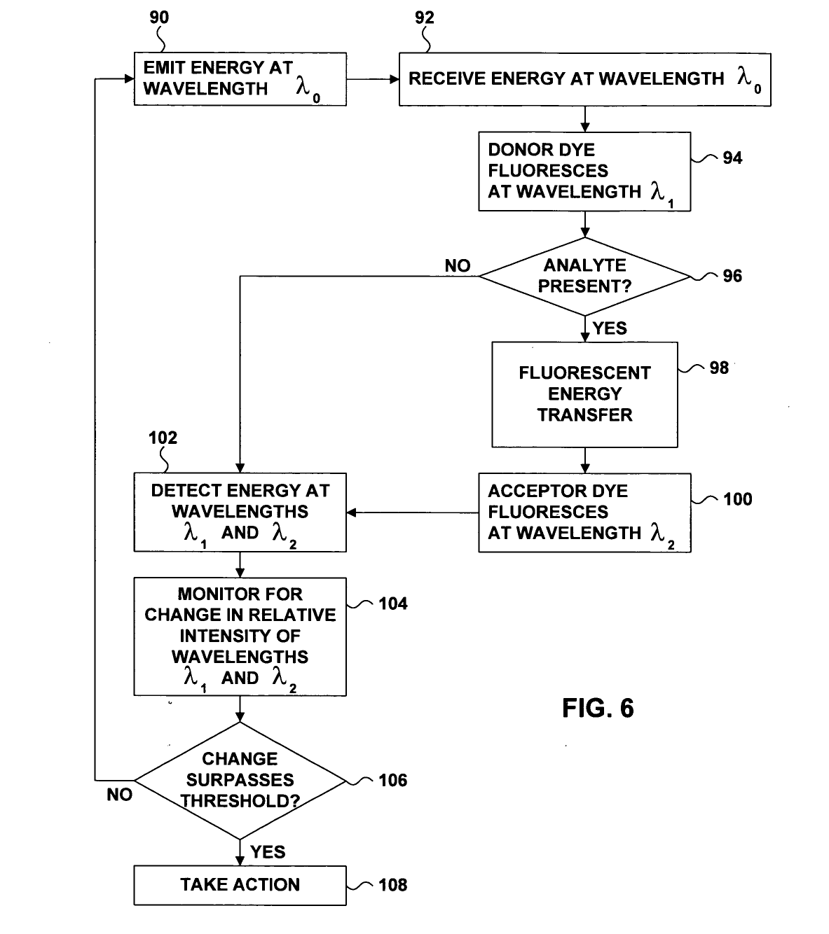

FIG. 6 is a flow diagram illustrating an embodiment of the invention. Light emitter 34 emits energy at wavelength 4 (90), which strikes analyte detector 32 (92). Analyte detector 32 includes a plurality of fluorophore-tagged sensing elements. A first dye on each fluorophore-tagged sensing element, donor fluorescent dye 70, fluoresces at wavelength λ₁ in response to the energy received from light emitter 34 (94). When an analyte of interest is present on a fluorophore-tagged sensing element (96), FRET takes place (98) and a second dye, acceptor fluorescent dye 72, fluoresces at wavelength λ₂ (100). When the analyte is not present on a fluorophore-tagged sensing element (96), FRET does not takes place. Analyte detector 32 often includes fluorophore-tagged sensing elements on which the analyte is present, and also includes fluorophore-tagged sensing elements on which the analyte is not present. Accordingly, analyte detector 32 fluoresces at wavelength λ₁ and wavelength λ₂.

Detector 36 detects the energy at wavelengths λ₁ and λ₂ (102). In particular, detector 36 responds to the intensities of the energy at wavelengths λ₁ and λ₂. Processor 38 receives the intensities and determines the relative intensity of the energy at wavelengths λ₁ and λ₂ with respect to one another by determining a ratio between the detected intensity of energy at wavelengths λ₁ and λ₂. Processor 38 further monitors the change in relative intensity over time (104). Processor 38 also compares detected changes in relative intensity to a threshold (106). Should the change surpass the threshold, processor 38 further may take action (108), such as communicating with therapy device 22 or notifying patient 10 via alert module 44.

A threshold that triggers action may be established experimentally, and may be a function of the analyte of interest. When the analyte is cardiac troponin-T antigen, for example, an increase in the intensity of energy at wavelength λ₂ relative to energy at wavelength λ₁ by a factor of two or more within an hour may cause processor 38 to take action. The threshold may be set by the patient's physician and may be stored in memory 40.

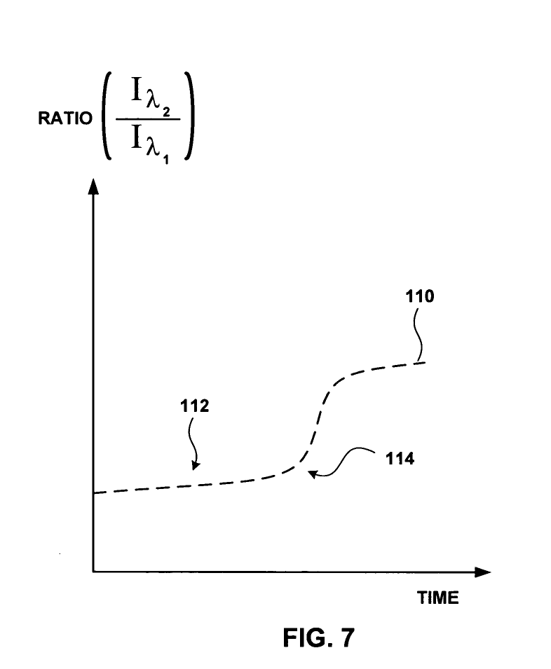

FIG. 7 is a timing diagram illustrating a curve 110 of relative intensity of the energy at wavelengths λ₁ and λ₂ over time. The relative intensity is expressed as a ratio I(λ₂)/I(λ₁). Each data point making up curve 110 may have been computed by processor 38 and stored in memory 40.

The relative intensity may change over time without surpassing a threshold and thereby prompting processor 38 to take action. A series of data points identified by reference numeral 112, for example, may show a steady increase in the ratio λ₂/λ₁, but such an increase is not a cause for concern. When the ratio I(λ₂)/I(λ₁) changes abruptly, as indicated by reference numeral 114, processor 38 may take action as described above.

The threshold may be defined in any number of ways, and the invention is not limited to any particular definition. In one embodiment, the threshold may be surpassed when the derivative of curve 110 exceeds a preselected value. In another embodiment, the threshold may be surpassed when the value of one data point exceeds the value of a previous data point by a preselected margin or a preselected percentage. In a further embodiment, the threshold may be surpassed when a ratio of two I(λ₂)/I(λ₁) ratios surpasses a preselected value.

The invention is not limited to any particular expression of relative intensity. For example, the relative intensity may also be expressed as a ratio λ₁/λ₂. In that case, the relative intensity may show a gradual decline over time without surpassing a threshold and thereby prompting processor 38 to take action. When the ratio drops abruptly, however, the threshold may be surpassed and processor 38 may take action as described above.

In one exemplary application of the invention, the invention may detect the presence or absence of an analyte of interest. In such a case, any substantial change in relative intensity may be sufficient to surpass the threshold and prompt processor 38 to take action.

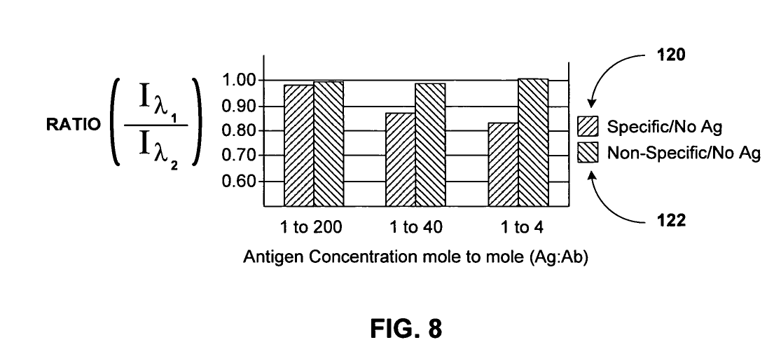

FIG. 8 is a bar chart illustrating specificity of analyte detection with FRET according to an embodiment of the invention. In the chart, relative intensity, expressed as a ratio I(λ₁)/I(λ₂), is a function of concentration. The chart of FIG. 8, which is based on actual data from an in vitro experiment, demonstrates specificity on a large scale.

The chart shows the response of an analyte sensor having sensing elements that include cardiac troponin-T antibodies. The cardiac troponin-T antibodies were generated by injection of cardiac troponin-T antigen into one or more mice as a foreign agent, and harvesting the cardiac troponin-T antibodies generated in response to the foreign agent.

The cardiac troponin-T antibodies were anchored to a substrate with Protein A. The sensing elements were fluorophore-tagged with FITC on the antibody and TRITC on Protein A.

The analyte sensor was illuminated with a light source having a wavelength within the excitation spectrum of FITC. Side-by-side bars show the response as a function of concentration of substances. One set of bars (120) depicts the response to the specific analyte of interest, cardiac troponin-T antigen, and the other set of bars (122) depicts the response to analytes other than cardiac troponin-T antigen. A ratio of 1.00 indicates no detection of cardiac troponin-T antigen. As shown in FIG. 8, increasing the concentration of substances other than cardiac troponin-T antigen failed to produce significant deviation from 1.00. Increasing the concentration of cardiac troponin-T antigen, on the other hand, produced significant changes to the ratio of intensity of detected wavelengths.

In other words, the in vitro data support the use of FRET to determine changes in concentration of a specific analyte, namely, cardiac troponin-T. The risks of false positives and false negatives are low. Similar in vitro data demonstrate that the same kind of analyte sensor can not only be specific to cardiac troponin-T, but can be sensitive to changes in concentration of cardiac troponin-T. Specificity and sensitivity for other analytes may be functions of the analyte in question. Specificity and sensitivity may also be a function of the sensitivity and noise-rejection capability of the light detector or processor in the analyte sensor.

The invention may provide one or more advantages. When using antibodies in sensing elements, the sensing elements can be highly specific to a particular analyte, and can be very sensitive to changes in concentration of the analyte. A fluorescence-based analyte detector may undergo some encapsulation by the body following implantation, but fluorescence-based detection is less sensitive to problems associated with encapsulation than other detection techniques. In addition, any encapsulation is likely to have comparable effects on all pertinent energy wavelengths, so the ratio between the detected intensity of energy at wavelengths λ₁ and λ₂ is not very sensitive to encapsulation. Moreover, encapsulation can be addressed by increasing the intensity of energy emitted wavelength λ₀. When processor 38 controls light emitter 34 to increase the intensity of energy emitted at wavelength λ₀, processor 38 may notify patient 10 via alert module 44 that the sensor may be due for maintenance or replacement.

Various embodiments of the invention have been described. Various modifications can be made to the described embodiments without departing from the scope of the invention. For example, although an analyte detector has been described as being specific to a single analyte, an analyte detector may also be specific to more than one analyte. A substrate may include a plurality of first sensing elements that are specific to a first analyte, and a plurality of second sensing elements that are specific to a second analyte. The different sensing elements may be tagged with different dyes. In addition, the invention encompasses embodiments in which the an analyte detector is implanted with the detector partially saturated with the analyte, which may improve the sensitivity and specificity of an analyte sensor.

As noted above, the invention is not limited to detection of troponin-T antigen, but may be adapted to detect other organic or inorganic analytes as well. For example, a sensor may be responsive to changes in concentration of D-dimers that accompany ischemic stroke. A patient that has had one ischemic stroke is at a high risk of having another ischemic stroke. A sensor that includes binding sites for D-dimers can respond to changes in concentration that accompany an ischemic stroke. Upon detection of a sudden increase in concentration, the sensor can take action, such as activation of a drug delivery device to deliver a blood thinner or a thrombolytic agent.

There are numerous other potential applications for the invention, and the invention is not limited to any particular application. For example, the invention may be used to detect the presence or absence of the Human Immunodeficiency Virus (HIV) or other virus. The invention may be used to track the concentrations of naturally occurring chemicals such as insulin or introduced medications such as beta-blockers. The invention may be used to detect illegal drugs, or food pathogens, or biological toxins, or biological warfare agents.

Similarly, the invention is not limited to sensing elements that include one or more antibody molecules. In general, the sensing element includes a binding site for a specific analyte, and the sensing element undergoes a conformational change that promotes FRET when the analyte binds to the binding site. The sensing element also is taggable with at least one fluorescent dye that does not interfere with the binding site. Sensing elements may include, but are not limited to, naturally occurring proteins and engineered proteins.

The invention may be embodied as a computer-readable medium that includes instructions for causing a programmable processor, such as processor 38 in FIGS. 2 and 3, to carry out the methods described above. A “computer-readable medium” includes but is not limited to read-only memory, Flash memory and a magnetic or optical storage medium. The instructions may be implemented as one or more software modules, which may be executed by themselves or in combination with other software. These and other embodiments are within the scope of the following claims.

1. A method comprising:
emitting energy at a first wavelength toward an analyte detector implanted in a body of a patient, the analyte detector including a binding site for an analyte, and the analyte detector including at least two fluorescent dyes;
detecting energy emitted by a first one of the dyes at a second wavelength based on fluorescent resonant energy transfer from a second one of the dyes in response to the emitted energy at the first wavelength; and
detecting a change in a concentration of the analyte as a function of the detected energy.

2. The method of claim 1, further comprising comparing the detected change to a threshold.

3. The method of claim 1, further comprising taking action when the detected change surpasses a threshold, wherein taking action comprises at least one of generating an alert and generating a communication to a therapy device.

4. The method of claim 1, further comprising detecting energy at a third wavelength emitted by the second one of the dyes in response to the emitted energy at the first wavelength.

5. The method of claim 4, further comprising determining a ratio between a detected intensity of the energy at the second wavelength and a detected intensity of energy at the third wavelength.

6. The method of claim 4, wherein detecting the change in concentration of the analyte comprises detecting a change in an intensity of the energy at the second wavelength relative to a detected intensity of energy at the third wavelength.

7. The method of claim 4, wherein the first one of the dyes absorbs energy received from the second one of the dyes at the third wavelength via fluorescent resonant energy transfer and emits energy at the second wavelength in response to receiving the energy at the third wavelength.

8. The method of claim 1, wherein the energy is visible light.

9. The method of claim 1, wherein the analyte comprises cardiac troponin-T antigen.

10. The method of claim 1, wherein the analyte comprises at least one of a D-dimer that accompanies an ischemic stroke, a virus, insulin, an administered medication, an illegal drug, a food pathogen, a biological toxin, and a biological warfare agent.

11. A system comprising:
a light emitter to emit energy at a first wavelength;
an analyte detector implanted in a body of a patient, the analyte detector including a binding site for an analyte and at least two fluorescent dyes;
a light detector to detect energy at a second wavelength emitted by a first one of the dyes based on fluorescent resonant energy transfer from a second one of the dyes in response to the emitted energy at the first wavelength; and
a processor to detect a change in a concentration of the analyte as a function of the energy detected by the light detector.

12. The system of claim 11, wherein at least one of the fluorescent dyes comprises fluorescein 5-isothiocyanate, tetramethylrhodamine 5-isothiocyanate, tetramethylrhodamine 6-isothiocyanate and aminomethylcoumarin acetate.

13. The system of claim 11, further comprising a therapy device to deliver therapy to the patient based on the detection of the change in a concentration.

14. The system of claim 13, wherein the therapy device comprises at least one of an implanted drug delivery device, a pacemaker and an electrical stimulator that stimulates at least one of an organ, a muscle and a nerve.

15. The system of claim 13, further comprising a communication module to generate a wireless communication from the processor to the therapy device.

16. The system of claim 13, wherein the processor is configured to compare the detected change to a threshold.

17. The system of claim 11, further comprising an alert module to notify the patient based on the detection of the change in a concentration.

18. The system of claim 11, wherein the a light emitter and the light detector are implanted in the body of the patient.

19. The system of claim 11, wherein the light detector is configured to detect energy at a third wavelength emitted by the second one of the dyes in response to the emitted energy at the first wavelength.

20. The system of claim 19, wherein the processor is configured to determine a ratio between a detected intensity of the energy at the second wavelength and a detected intensity of energy at the third wavelength.

21. The system of claim 19, wherein the processor is configured to detect the change in concentration of the analyte by detecting a change in an intensity of the energy at the second wavelength relative to a detected intensity of energy at the third wavelength.

22. A device comprising:
a substrate;
two fluorescent dyes; and
a plurality of sensing elements anchored to the substrate, each sensing element including a binding site for an analyte, and each sensing element configured to bring the dyes into proximity to allow fluorescent resonant energy transfer when the analyte binds to the binding site.

23. The device of claim 22, wherein at least one of the fluorescent dyes comprises fluorescein 5-isothiocyanate, tetramethylrhodamine 5-isothiocyanate, tetramethylrhodamine 6-isothiocyanate and aminomethylcoumarin acetate.

24. The device of claim 22, further comprising an anchoring agent to anchor at least one sensing element to the substrate.

25. The device of claim 24, wherein the anchoring agent comprises Protein A.

26. The device of claim 24, wherein the substrate comprises at least one of silicone and glass.

27. The device of claim 22, wherein at least one sensing element comprises an antibody molecule.

28. The device of claim 27, wherein the antibody molecule comprises a Troponin-T antibody molecule.

29. The device of claim 22, wherein the substrate comprises a permeable material to anchor the sensing element to the substrate.

30. The device of claim 29, wherein the permeable material comprises at least one of a hydrogel and a matrix of polytetrafluoroethylene.

31. The device of claim 22, wherein the substrate includes a surface having ridges and grooves.

32. The device of claim 22, wherein the device is implantable in a patient.

33. A computer-readable medium comprising instructions for causing a programmable processor to:
control a light emitter to emit energy at a first wavelength toward an analyte detector implanted in a body of a patient, the analyte detector including a binding site for an analyte, and the analyte detector including at least two fluorescent dyes;
receive a signal from a detector as a function of energy emitted by a first one of the dyes at a second wavelength based on fluorescent resonant energy transfer from a second one of the dyes in response to the emitted energy at the first wavelength; and
detecting a change in a concentration of the analyte as a function of the received signal.

34. The medium of claim 33, the instructions further causing the processor to compare the detected change to a threshold.

35. The medium of claim 33, the instructions further causing the processor to take action when the detected change surpasses a threshold, wherein taking action comprises at least one of notifying a patient and generating a communication to a therapy device.

36. The medium of claim 33, the instructions further causing the processor to receive a signal from the detector as a function of energy at a third wavelength emitted by the second one of the dyes in response to the emitted energy at the first wavelength.

37. The medium of claim 36, the instructions further causing the processor to determine a ratio between a detected intensity of the energy at the second wavelength and a detected intensity of energy at the third wavelength.
